(19) United States
(12) Patent Application Publication
(10) Pub. No.: US 2005/0008596 A1
(43) Pub. Date: Jan. 13, 2005

(54) Composition containing an oxidation-sensitive active principle and a polyisobutylene polymer

(75) Inventors: Bruno Biatry, Vincennes (FR); Jean-Thierry Simonnet, Cachan (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/849,159

(22) Filed: May 20, 2004

Related U.S. Application Data
(60) Provisional application No. 60/489,921, filed on Jul. 25, 2003.

(30) Foreign Application Priority Data
May 27, 2003 (FR) 0306421

(51) Int. Cl. A61K 7/06; A61K 7/11

(52) U.S. Cl. 424/70.11

(57) ABSTRACT

The invention relates to a composition containing at least one hydrophilic active principle and at least one amphiphilic polyisobutylene polymer. The invention also relates to the use of such a polymer for stabilizing an oxidation-sensitive hydrophilic active principle, in particular in the aqueous phase. The present invention also relates to the uses of this composition.

### REFERENCE TO PRIOR APPLICATIONS

This application claims priority to U.S. provisional application 60/489,921 filed Jul. 25, 2003, and to French patent application 0306421 filed May 27, 2003, both incorporated herein by reference.

### FIELD OF THE INVENTION

The present invention relates to a composition, preferably a cosmetic and/or dermatological composition for topical use, comprising at least one hydrophilic active principle (for example one selected from the group consisting of ascorbic acid and its derivatives—i.e., an ascorbic acid compound), and at least one amphiphilic polymer selected from the group consisting of oligomers and polymers derived from polyisobutylene (i.e., a polyisobutylene polymer).

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is known practice to introduce into cosmetic compositions various active principles intended to contribute specific treatments to the skin and/or the hair. However, some of these active principles have the drawback of being unstable in an aqueous medium and of readily decomposing on contact with water, in particular because of oxidation phenomena. They thus rapidly lose their activity over time and this instability conflicts with the desired effectiveness.

Attempts have thus been made for a long time to formulate ascorbic acid or vitamin C, because of its many beneficial properties. In particular, ascorbic acid stimulates the synthesis of connective tissue, and in particular of collagen, strengthens skin tissue defences against external attacks, such as ultraviolet radiation and pollution, compensates for vitamin E deficiency of the skin, and has a free-radical scavenger function. These last two properties make it an excellent candidate as a cosmetic or dermatological active principle for combating ageing of the skin or for preventing it. Moreover, ascorbic acid possesses a depigmenting activity on the skin by blocking melanogenesis by reduction of DOPA quinone.

Unfortunately, because of its chemical structure (of alpha-ketolactone), ascorbic acid is highly sensitive to certain environmental parameters, and in particular to oxidation phenomena. This ensures rapid decomposition of formulated ascorbic acid in the presence of these parameters, and more particularly in the presence of oxygen, light or metal ions, as a function of the temperature or under certain pH conditions (Pharm. Acta. Helv., 1969, 44, 611-667; STP Pharma, 1985, 4, 281-286).

Several solutions have therefore been envisaged in the prior art for reducing or slowing down the decomposition of ascorbic acid.

It has thus been proposed to use ascorbic acid in the form of a chemical derivative (magnesium ascorbyl phosphate or esters of fatty acids and ascorbic acid), but the bioavailability of these derivatives is very low (J. Am. Acad. Dermatol., 1996, 34, 29-33).

It has been possible to improve the instability of ascorbic acid with respect to oxygen by using specific packaging, such as twin compartments under an inert atmosphere, as disclosed in U.S. Pat. No. 5,935,584, or alternatively by using two-phase emulsions, one phase of which consists of a dry powder containing ascorbic acid and the second phase of which consists of a liquid phase. The mixing of the two phases has to be carried out at the time of use (WO 98/43598). These solutions have disadvantages in terms of cost and of the complexity of the manufacturing operations, and also significant restrictions with regard to use.

Another solution provided in the prior art consists in using a high concentration of glycols or polyols in order to reduce the solubility of oxygen in the formulation, thus protecting the ascorbic acid (WO 96/24325, EP 0 755 674, U.S. Pat. No. 5,981,578). The polyols can optionally be incorporated into liposomes, as described in U.S. Pat. No. 6,020,367. However, these solutions have the disadvantage of resulting in sticky formulations, the cosmetic quality of which is difficult to improve. Moreover, the presence of a high concentration of these compounds can lead to phenomena of irritation. Since the solubility of ascorbic acid in these polyols is insufficient, it is sometimes necessary to use solubilizing agents such as N-alkylpyrrolidones (WO 00/78283) or avocado oil derivatives (U.S. Pat. No. 5,981,578), which makes the formulation process more complex.

Ascorbic acid can also be formulated in anhydrous media, such as silicones (U.S. Pat. No. 6,194,452), which are capable of creating an anhydrous barrier around the ascorbic acid. A major disadvantage of such solutions results from the lack of freshness on application.

### SUMMARY OF THE INVENTION

The need thus remains for a composition which can be used in particular in the cosmetics field, in which a hydrophilic active principle which is unstable in oxidizing medium is stabilized, which is comfortable on application, which does not lead to any skin irritation after application, and which is compatible with the constraints of an industrial implementation of its manufacturing process. The present invention provides such a composition.

### DETAILED DESCRIPTION OF THE PREFERRED EMBODIMENTS

The present invention provides a composition containing an oxidation-sensitive active principle which preferably exhibits good cosmetic properties, both with regard to touch and with regard to tolerance, the preservation of which over time does not require specific precautions.

The inventors have discovered, fortuitously, that the use of certain amphiphilic polymers selected from the group consisting of oligomers and polymers derived from polyisobutylene comprising an apolar polyisobutylene component and at least one terminal polar component, in compositions in which the aqueous phase contains an oxidation-sensitive active principle, such as ascorbic acid, makes it possible to achieve the abovementioned aim.

Oligomers and polymers derived from polyisobutylene are known in other fields. Thus, they are described as stabilizers of explosive emulsions, which are invert emulsions of molten ammonium nitrate or of a saturated solution of ammonium nitrate in a hydrocarbon-based oil. Moreover, these compounds are known as stabilizers of fertilizing compositions in the form of a water-in-oil emulsion (see document U.S. Pat. No. 5,518,517) with a view to obtaining controlled release of the fertilizing substances. In addition, in Application EP-1 172 089, compositions, in particular cosmetic compositions, in the form of W/O emulsions containing the abovementioned compounds have been described. These compounds are stable and fresh on application.

In the W/O emulsions described in those documents, the aqueous internal phase contains substances specific to the field of application of the composition, solubilized or dispersed in water. No mention is, however, made of ascorbic acid or of its derivatives. In addition, it is not suggested in those documents that the oligomers and polymers derived from polyisobutylene have any stabilizing power with respect to oxidation-sensitive hydrophilic active principles such as ascorbic acid and its derivatives.

One subject of the present invention is therefore a composition for topical use, comprising, in a physiologically acceptable medium, at least one hydrophilic active principle selected from the group consisting of ascorbic acid and its derivatives (i.e., an ascorbic acid compound), and at least one amphiphilic polymer selected from the group consisting of the oligomers or polymers derived from polyisobutylene (i.e., a polyisobutylene polymer) comprising an apolar polyisobutylene component comprising at least 40 carbon atoms, and at least one terminal polar component selected from the group consisting of carboxylic acids and diacids, of anhydrides thereof or of modified forms thereof in the form of esters, of amides or of salts, and mixtures thereof, said composition being devoid of N-vinylimidazole polymer or copolymer. The amphiphilic polymer is preferably present in an amount sufficient to stabilize said hydrophilic active principle with respect to oxidation.

The term “N-vinylimidazole polymer or copolymer” is intended to mean any polymer or copolymer comprising one or more N-vinylimidazole units.

The invention also relates to the use of the invention polyisobutlyene for stabilizing an oxidation-sensitive hydrophilic active principle, for example by bringing the two materials together in the same composition in any order, adding the two materials to the same composition in any order, etc (i.e., contacting the two materials).

The use of the at least one amphiphilic polymer selected from the group consisting of the oligomers or polymers derived from polyisobutylene comprising an apolar polyisobutylene component comprising at least 40 carbon atoms, and at least one terminal polar component selected from the group consisting of carboxylic acids and diacids, of anhydrides thereof or of modified forms thereof in the form of esters, of amides or of salts, and mixtures thereof (i.e., the preferred polyisobutlyene) for stabilizing an oxidation-sensitive hydrophilic active principle has the advantage of being able to stabilize this active principle in compositions having, without limitation, a pH between 5 and 7, i.e. under conditions which respect the physiological equilibrium of the skin, the pH of which is in the region of 5.5.

According to the invention, the term “hydrophilic active principle” is intended to mean a compound having a water-solubility of at least 0.25% at ambient temperature (25° C.).

According to the invention, the expression “oxidation-sensitive hydrophilic active principle” is intended to mean any active principle of natural or synthetic origin capable of undergoing decomposition via an oxidation mechanism. This oxidation phenomenon can have several causes, in particular the presence of oxygen, light or metal ions, a high temperature, or else certain pH conditions.

By way of example, and in a nonlimiting manner, mention may be made of: ascorbic acid and its derivatives (i.e., an ascorbic acid compound), such as 5,6-di-O-dimethylsilylascorbate (sold by the company Exsymol under the reference PRO-AA), the potassium salt of dl-alpha-tocopheryl-dl-ascorbyl phosphate (sold by the company Senju Pharmaceutical under the reference Sepivital EPC), magnesium ascorbyl phosphate, sodium ascorbyl phosphate (sold by the company Roche under the reference Stay-C 50) and ascorbyl glucoside (sold by the company Hayashibara).

Among the hydrophilic active principles sensitive to oxidation and/or derived from ascorbic acid, preference will be given more particularly to ascorbic acid itself.

The use of the amphiphilic polymers selected from the group consisting of the oligomers or polymers derived from polyisobutylene according to the invention, for stabilizing an oxidation-sensitive hydrophilic active principle, is particularly suitable for stabilizing said active principle at a concentration of between 0.5 and 20% by weight, preferably between 1 and 15% by weight, relative to the total weight of the composition.

Preferred amphiphilic polymers selected from the group consisting of the oligomers and polymers (which may be thought of as being used as emulsifiers in the composition of the invention) generally include an apolar polyisobutylene component and at least one polar component.

The apolar polyisobutylene component preferably comprises at least 40 carbon atoms, and more preferably from 60 to 700 carbon atoms. It is highly preferred for this component to comprise at least 40 carbon atoms in order to best achieve the aim of the invention. If there are fewer than 40 carbon atoms, the stability of the system may be lessened.

The polar component of the oligomeric or polymeric emulsifiers of the invention is preferably selected from carboxylic acids or diacids, anhydrides thereof or modified forms thereof in the form of esters, amides or salts, and mixtures thereof. Preferably, the terminal polar component consists of carboxylic diacids or of anhydrides thereof or of modified forms thereof in the form of esters, of amides or of salts.

The expression “modified forms in the form of esters, of amides or of salts” denotes carboxylic acids or diacids modified with alcohols, amines, alkanolamines or polyols, or in the form of, e.g., alkali metal or alkaline earth metal salts, of ammonium salts or else of salts of an organic base, such as diethanolamine and triethanolamine salts.

Oligomeric or polymeric emulsifiers containing a carboxylic acid polar component may, for example, be derived from the reaction between a polyisobutylene and at least one carboxylic acid or anhydride selected from the group consisting of the group comprising maleic acid, maleic anhydride, fumaric acid, itaconic acid, citraconic acid, mesaconic acid and aconitic acid. Preferably, the polar component consists of succinic acid or anhydride, the modified ester or amide forms thereof, or the corresponding alkali metal or alkaline earth metal ion salts or organic salts, it being possible for these compounds to be optionally polyoxyethylenated.

The emulsifiers derived from succinic acid or anhydride can be chosen in particular from the polyisobutylene derivatives of succinic acid or anhydride described in U.S. Pat. No. 4,234,435, U.S. Pat. No. 4,708,753, U.S. Pat. No. 5,129,972, U.S. Pat. No. 4,93 1,110, GB-A-2,156,799 and U.S. Pat. No. 4,919,179. The polyisobutylene component may or may not be hydrogenated, for example with a molecular weight ranging from 400 to 5000. In the polyisobutylene containing a succinic end group thus obtained, the succinic component can be esterified, amidated or in the form of a salt, i.e. it can be advantageously modified with alcohols, amines, alkanolamines or polyols, or else can be in the form of alkali metal or alkaline earth metal salts, ammonium salts or else salts of an organic base, such as diethanolamine and triethanolamine salts. The polyisobutylenes containing a succinic end group which is esterified or amidated are products of the reaction of (a) a polyisobutylene with a succinic end group, and of (b) an amine or an alcohol, to fonn an amide or an ester. The term “amine” used here comprises all types of amines, including alkanolamines. This may include, for example, primary, secondary or tertiary amines, it being possible for these amines to be aliphatic, cycloaliphatic, aromatic, heterocyclic, saturated or unsaturated. Moreover, the alcohols may be mono- or polyalcohols. The monoalcohols comprise primary, secondary or tertiary aliphatic alcohols, and phenols. The polyalcohols can, for example, be selected from the group consisting of aliphatic, cycloaliphatic, aromatic and heterocyclic polyalcohols. Polyisobutylenes with a modified (esterified or amidated) succinic end group and the method for preparing them are described in particular in document U.S. Pat. No. 4,708,753.

As polyisobutylene containing a succinic end group, mention may in particular be made of polyisobutylenes with a modified succinic end group, such as the products marketed under the names Lubrizol 5603 and Lubrizol 2650 by the company Lubrizol. According to a preferred embodiment of the invention, use is made of the polymer marketed under the name Lubrizol 5603 by the company Lubrizol, which is the diethylethanolamine salt of polyisobutylene containing an esterified succinic end group (INCI name: hydroxyethyldiethonium polyisobutenyl triethylaminosuccinate/ diethylethanolamine).

Another example of a polyisobutylene derivative which can be used in the invention is the product of the reaction of maleic anhydride with polyisobutylene, such as the product marketed under the name Glissopal SA by the company BASF.

The amount of aqueous phase in the compositions according to the invention is not limited and preferably is at least 40% by weight relative to the total weight of the composition. It may range, for example, from 40 to 95% by weight, preferably from 50 to 90%, better still from 60 to 90% by weight, and even better still from 80 to 90% by weight, relative to the total weight of the composition. The composition of the invention preferably contains at least 30% by weight of water, and better still at least 50% by weight of water, relative to the total weight of the composition.

The amount of oligomer(s) or of polymer(s) derived from polyisobutylene in the composition of the invention is not limited and may range, for example, from 0.1 to 10% by weight of active material, preferably from 0.5 to 5% by weight, and better still from 1 to 3% by weight, relative to the total weight of the composition. One or more oligomers or polymers derived from polyisobutylene may be used.

In the compositions according to the present invention, the molar ratio of the oligomer or polymer derived from polyisobutylene to the hydrophilic active principle is not limited but preferably ranges between 0.0006 and 3, more preferably between 0.001 and 0.1, and more particularly between 0.01 and 0.5.

According to a preferred embodiment of the invention, the oligomers or polymers derived from polyisobutylene according to the invention are the only emulsifiers used in the composition according to the invention.

However, it is also possible to optionally add other amphiphilic agents, in particular those used in water-in-oil emulsions, such as conventional ionic, nonionic, amphoteric or zwitterionic surfactants, amphiphilic oligomers or polymers, or amphiphilic inorganic or organic particles.

The compositions used according to the invention are preferably intended for topical application to the skin and/or its superficial body growths and therefore preferably contain a physiologically acceptable medium, i.e. a medium compatible with cutaneous tissues such as the skin, scalp, eyelashes, eyebrows, hair, nails and mucous membranes. This physiologically acceptable medium may more particularly be or comprise water and, optionally, one or more physiologically acceptable organic solvents chosen, for example, from lower alcohols containing from 1 to 8 carbon atoms, and in particular from 1 to 6 carbon atoms, such as ethanol, isopropanol, propanol or butanol; polyethylene glycols having from 6 to 80 ethylene oxide units; or polyols such as polypropylene glycol, isoprene glycol, butylene glycol, glycerol or sorbitol.

This physiologically acceptable medium is preferably a cosmetically acceptable medium, i.e. it has a pleasant appearance, odour and colour and does not generate discomfort (tingling, tightness, red blotches) unacceptable to the user. When the physiologically acceptable medium is an aqueous medium, it thus generally has a pH which is compatible with the skin, preferably ranging from 3 to 9, and better still from 3.5 to 7.5.

The compositions according to the invention can be provided in any form, for exsample forms conventionally used for topical application, and in particular in the form of aqueous or aqueous-alcoholic solutions, of oil-in-water (O/W) or water-in-oil (W/O) or multiple (triple: W/O/W or O/W/O) emulsions, of aqueous gels or of dispersions of a fatty phase in an aqueous phase using spherules, it being possible for these spherules to be polymeric nanoparticles, such as nanospheres or nanocapsules, or lipid vesicles of ionic and/or nonionic type (liposomes, niosomes or oleosomes). These compositions may be prepared according to known methods by one of ordinary skill in view of the present disclosure.

In addition, the compositions used according to the invention can be more or less fluid and can have the appearance of a white or coloured cream, of an ointment, of a milk, of a lotion, of a serum, of a paste or of a foam. They can optionally be applied to the skin in the form of an aerosol. They can also be provided in solid form, for example in the form of a stick.

When the composition used according to the invention comprises an oily phase, this phase preferably contains at least one oil. It can also contain other fatty substances.

Mention may be made, as oils which can be used in the composition of the invention, of, for example:
hydrocarbon-based oils of animal origin, such as perhydrosqualene;
hydrocarbon-based oils of plant origin, such as liquid triglycerides of fatty acids containing from 4 to 10 carbon atoms, such as triglycerides of heptanoic or octanoic acid, or else, for example, sunflower oil, corn oil, soybean oil, gourd oil, grapeseed oil, sesame oil, hazelnut oil, apricot oil, macadamia oil, arara oil, castor oil or avocado oil, triglycerides of caprylic/capric acids, such as those sold by the company Stearineries Dubois or those sold under the names Miglyol 810, 812 and 818 by the company Dynamit Nobel, jojoba oil or karite butter oil;
synthetic esters and ethers, in particular of fatty acids, such as oils of formulae R¹COOR² and R¹OR² in which R¹ represents the residue of a fatty acid containing from 8 to 29 carbon atoms, and R² represents a branched or unbranched hydrocarbon-based chain containing from 3 to 30 carbon atoms, such as, for example, Purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;
linear or branched hydrocarbons of mineral or synthetic origin, such as volatile or nonvolatile liquid paraffins and their derivatives, petroleum jelly, polydecenes or hydrogenated polyisobutene, such as parleam oil;
fatty alcohols having from 8 to 26 carbon atoms, such as cetyl alcohol, stearyl alcohol and their mixture (cetearyl alcohol), octyldodecanol, 2-butyl-octanol, 2-hexyldecanol, 2-undecylpentadecanol, oleyl alcohol or linoleyl alcohol;
partially hydrocarbon-based and/or silicone-based fluoro oils such as those described in document JP-A-2-295912;
silicone oils, such as volatile or nonvolatile polymethylsiloxanes (PDMSs) containing a linear or cyclic silicone chain, which are liquid or pasty at ambient temperature, in particular cyclopolydimethylsiloxanes (cyclomethicones), such as cyclohexasiloxane; polydimethylsiloxanes comprising alkyl, alkoxy or phenyl groups, pendant or at the end of the silicone chain, which groups have from 2 to 24 carbon atoms; phenyl silicones, such as phenyl trimethicones, phenyl dimethicones, phenyltrimethylsiloxydiphenylsiloxanes, diphenyl dimethicones, diphenylmethyldiphenyltrisiloxanes, (2-phenylethyl)trimethylsiloxysilicates, and polymethylphenylsiloxanes;
mixtures thereof.

The term “hydrocarbon-based oil” is intended to mean, in the list of oils mentioned above, any oil predominantly comprising carbon and hydrogen atoms and, optionally, ester, ether, fluoro, carboxylic acid and/or alcohol groups.

The other fatty substances which can be present in the oily phase are, for example, fatty acids containing from 8 to 30 carbon atoms, such as stearic acid, lauric acid, palmitic acid and oleic acid; waxes such as lanolin, beeswax, carnauba or candelilla wax, paraffin or lignite waxes or microcrystalline waxes, ceresin or ozokerite, or synthetic waxes such as polyethylene waxes or Fischer-Tropsch waxes; silicone resins such as trifluoromethyl-C₁-4-alkyl dimethicone and trifluoropropyl dimethicone; and silicone elastomers such as the products marketed under the names “KSG” by the company Shin-Etsu, under the names “Trefil”, “BY29” or “EPSX” by the company Dow Corning, or under the names “Gransil” by the company Grant Industries.

These fatty substances can be chosen in a varied manner by those skilled in the art in order to prepare a composition having the desired properties, for example of consistency or of texture.

According to a particular embodiment of the invention, the composition according to the invention is a water-in-oil (W/O) or oil-in-water (O/W) emulsion. The proportion of the oily phase of the emulsion can range for example from 5 to 80% by weight, and preferably from 5 to 50% by weight, relative to the total weight of the composition.

In an advantageous aspect of the invention, the compositions are provided in the form of water-in-oil emulsions.

In the case of the water-in-oil emulsions, the proportion of the aqueous phase in the emulsion is preferably at least 40% by weight relative to the total weight of the composition. It can in particular be between 40 and 95% by weight, preferably between 50 and 90% by weight, more particularly between 60 and 90% by weight, and most particularly between 80 and 90% by weight, relative to the total weight of the composition.

The emulsions can also contain at least one emulsifier selected from the group consisting of amphoteric, anionic, cationic or nonionic emulsifiers, used alone or as a mixture, and optionally a co-emulsifier. The emulsifiers are chosen in an appropriate manner according to the emulsion to be obtained (W/O or O/W). The emulsifier and the co-emulsifier are generally present in the composition in a proportion ranging from 0.3 to 30% by weight, and preferably from 0.5 to 20% by weight, relative to the total weight of the composition.

For the W/O emulsions, mention may be made, for example, as emulsifiers, of dimethicone copolyols, such as the mixture of cyclomethicone and of dimethicone copolyol sold under the name “DC 5225 C” by the company Dow Corning, and alkyl dimethicone copolyols, such as the laurylmethicone copolyol sold under the name “Dow Corning 5200 Formulation Aid” by the company Dow Corning and the cetyl dimethicone copolyol sold under the name Abil EM 90^{R} by the company Goldschmidt. Use may also be made, as a surfactant of W/O emulsions, of a crosslinked solid organopolysiloxane elastomer comprising at least one oxyalkylenated group, such as those obtained according to the procedure of Examples 3, 4 and 8 of document U.S. Pat. No. 5,412,004 and of the examples of document U.S. Pat. No. 5,811,487, in particular the product of Example 3 (synthesis example) of U.S. Pat. No. 5,412,004, and such as that marketed under the reference KSG 21 by the company Shin Etsu. It is also possible to use, as co-emulsifier, the disodium tetrapropenyl disuccinate sold under the name Rewocoros B3010® by the company Witco.

For the O/W emulsions, mention may be made, for example, as emulsifiers, of nonionic emulsifiers, such as esters of fatty acids and of glycerol which are oxyalkylenated (more particularly polyoxyethylenated); esters of fatty acids and of sorbitan which are oxyalkylenated; esters of fatty acids which are oxyalkylenated (oxyethylenated and/or oxypropylenated); ethers of fatty alcohols which are oxyalkylenated (oxyethylenated and/or oxypropylenated); sugar esters such as sucrose stearate; and mixtures thereof such as the mixture of glyceryl stearate and of PEG-40 stearate.

In a known way, the cosmetic or dermatological composition of the invention can also contain adjuvants that are conventional in the cosmetics or dermatological field, such as hydrophilic or lipophilic gelling agents, preserving agents, solvents, fragrances, fillers, UV-screening agents, bactericides, odour absorbers, dyestuffs, plant extracts or salts. The amounts of these various adjuvants are those conventionally used in the field under consideration, for example from 0.01 to 20% of the total weight of the composition. Depending on their nature, these adjuvants can be introduced into the fatty phase, into the aqueous phase and/or into the lipid spherules.

As fillers which can be used in the composition of the invention, mention may, for example, be made of, besides pigments, silica powder; talc; particles of polyamide and in particular those sold under the name Orgasol by the company Atochem; polyethylene powders; microspheres based on acrylic copolymers, such as those made of ethylene glycol dimethacrylate/lauryl methacrylate copolymer which are sold by the company Dow Corning under the name Polytrap; expanded powders, such as hollow microspheres and in particular the microspheres marketed under the name Expancel by the company Kemanord Plast or under the name Micropearl F 80 ED by the company Matsumoto; silicone resin microbeads such as those marketed under the name Tospearl by the company Toshiba Silicone; and mixtures thereof. These fillers can be present in amounts ranging from 0 to 20% by weight, and preferably from 1 to 10% by weight, relative to the total weight of the composition.

According to a preferred embodiment of the invention, the composition used according to the invention contains at least one UV-screening agent (or sunscreen) which may be a chemical screening agent or a physical screening agent or a mixture of such screening agents.

By way of illustration and in a nonlimiting manner, mention may be made of the following families (the names correspond to the CTFA nomenclature for screening agents):
anthranilates, in particular menthyl anthranilate; benzophenones, in particular benzophenone-1, benzophenone-3, benzophenone-5, benzophenone-6, benzophenone-8, benzophenone-9, benzophenone- 12, and preferably benzophenone-2 (oxybenzone), or benzophenone-4 (Uvinul MS40 available from BASF); benzylidenecamphors, in particular 3-benzylidenecamphor, benzylidenecamphorsulphonic acid, camphor benzalkonium methosulphate, polyacrylamidomethylbenzylidenecamphor, terephthalylidenedicamphorsulphonic acid, and preferably 4-methylbenzylidenecamphor (Eusolex 6300 available from Merck); benzimidazoles, in particular benzimidazilate (Neo Heliopan AP available from Haarmann and Reimer) or phenylbenzimidazolesulphonic acid (Eusolex 232 available from Merck); benzotriazoles, in particular drometrizole trisiloxane, or methylenebis(benzotriazolyl)tetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate, isoamyl cinnamate, and preferably ethocrylene (Uvinul N35 available from BASF), octyl methoxycinnamate (Parsol MCX available from Hoffmann La Roche) or octocrylene (Uvinul 539 available from BASF); dibenzoylmethanes, in particular butyl methoxydibenzoylmethane (Parsol 1789); imidazolines, in particular ethylhexyldimethoxybenzylidenedioxoimidazoline; PABAs, in particular ethyl dihydroxypropyl PABA, ethylhexyl dimethyl PABA, glyceryl PABA, PABA, PEG-25 PABA, and preferably
diethylhexylbutamidotriazone (Uvasorb HEB available from 3V Sigma), ethylhexyltriazone (Uvinul T150 available from BASF), or ethyl PABA (benzocaine); salicylates, in particular dipropylene glycol salicylate, ethylhexyl salicylate, homosalate, or TEA salicylate; triazines, in particular anisotriazine (Tinosorb S available from Ciba); drometrizole trisiloxane, zinc oxide and titanium dioxide.

The amount of screening agents depends on the desired final use. It can range, for example, from 1 to 20% by weight, and better still from 2 to 10% by weight, relative to the total weight of the composition.

According to another embodiment of the invention, the composition used also contains at least one active principle selected from the group consisting of depigmenting agents, NO-synthase inhibitors, agents for stimulating the synthesis of dermal or epidermal macromolecules and/or for preventing their degradation, agents for stimulating the proliferation of fibroblasts and/or keratinocytes and/or for stimulating keratinocyte differentiation, myorelaxants, tensioning agents and anti-pollution agents or free-radical scavengers.

The depigmenting agents which can be incorporated into the composition according to the present invention comprise, for example, the following compounds: cojic acid; ellagic acid; arbutin and its derivatives such as those described in Applications EP-895 779 and EP-524 109; hydroquinone; aminophenol derivatives such as those described in Applications WO 99/10318 and WO 99/32077, and in particular N-cholesteryloxycarbonyl-para-aminophenol and N-ethyloxycarbonyl-para-aminophenol; iminophenol derivatives, in particular those described in Application WO 99/22707; L-2-oxothiazolidine-4-carboxylic acid or procysteine, and also its salts and esters; plant extracts, in particular extracts of liquorice, of mulberry and of skullcap, without this list being limiting.

Examples of NO-synthase inhibitors that are suitable for use in the present invention comprise in particular an extract of a plant of the species Vitis vinifera which is in particular marketed by the company Euromed under the name Leucocyanidines de raisins extra, or by the company Indena under the name Leucoselect®, or finally by the company Hansen under the name Extrait de marc de raisin; an extract of a plant of the species Olea europaea which is preferably obtained from olive tree leaves and is in particular marketed by the company Vinyals in the form of a dry extract, or by the company Biologia & Technologia under the trade name Eurol BT; and an extract of a plant of the species Gingko biloba which is preferably a dry aqueous extract of this plant sold by the company Beaufour under the trade name Gingko biloba extrait standard.

Among the active principles for stimulating dermal macromolecules or for preventing a degradation thereof, mention may be made of those which act:
either on collagen synthesis, such as extracts of Centella asiatica; asiaticosides and derivatives; synthetic peptides such as iamin, biopeptide CL or palmitoyloligopeptide sold by the company Sederma; peptides extracted from plants, such as the soybean hydrolysate marketed by the company Coletica under the trade name Phytokine®; and plant hormones such as auxins;
or on elastin synthesis, such as the extract of Saccharomyces cerevisiae marketed by the company LSN under the trade name Cytovitin®; and the extract of the alga Macrocystis pyrifera marketed by the company Secma under the trade name Kelpadelie®;
or on glycosaminoglycan synthesis, such as the product of fermentation of milk by lactobacillus vulgaris, marketed by the company Brooks under the trade name Biomin yogourth®; the extract of the brown alga Padina pavonica marketed by the company Alban Müller under the trade name HSP3®; and the extract of Saccharomyces cerevisiae available in particular from the company Silab under the trade name Firmalift® from the company LSN under the trade name Cytovitin®;
or on fibronectin synthesis, such as the extract of the zooplankton Salina sold by the company Seporga under the trade name GP4G®; the yeast extract available in particular from the company Alban Müller under the trade name Drieline®; and the palmitoyl pentapeptide marketed by the company Sederma under the trade name Matrixil®;
or on the inhibition of metalloproteinases (MMPs) such as more particularly MMP 1, 2, 3 or 9. Mention may be made of: retinoids and derivatives, oligopeptides and lipopeptides, lipoamino acids, the malt extract marketed by the company Coletica under the trade name Collalift®; extracts of blueberry or of rosemary; lycopene; isoflavones, their derivatives or the plant extracts containing them, in particular the extracts of soybean (sold, for example, by the company Ichimaru Pharcos under the trade name Flavosterone SB®) of red clover, of flax, of kakkon or of sage;
or on the inhibition of serine proteases such as leucocyte elastase or cathepsin G. Mention may be made of: the peptide extract of legume seeds (Pisum sativum) marketed by the company LSN under the trade name Parelastyl®; heparinoids; and pseudodipeptides such as {2-[acetyl-(3-trifluoromethylphenyl) amino]-3-methylbutyrylamino} acetic acid.

Among the active principles for stimulating epidermal macromolecules, such as fillagrin or keratins, mention may in particular be made of the extract of lupin marketed by the company Silab under the trade name Structurine®; the extract of beech Fagus sylvatica buds marketed by the company Gattefosse under the trade name Gatuline®; and the extract of the zooplankton Salina marketed by the company Seporga under the trade name GP4G®.

The agents for stimulating the proliferation of fibroblasts which can be used in the composition according to the invention can, for example, be selected from the group consisting of plant proteins or polypeptides, extracts, in particular of soybean (for example an extract of soybean marketed by the company LSN under the name Eleseryl SH-VEG 8® or marketed by the company Silab under the trade name Raffermine®); and plant hormones such as giberrellins and cytokinins.

The agents for stimulating the proliferation of keratinocytes which can be used in the composition according to the invention comprise in particular retinoids such as retinol and its esters, including retinyl palmitate; phloroglucinol; extracts of nut cakes marketed by the company Gattefosse; and extracts of Solanum tuberosum marketed by the company Sederma.

The agents for stimulating keratinocyte differentiation comprise, for example, minerals such as calcium; the extract of lupin marketed by the company Silab under the trade name Photopreventine®; sodium beta-sitosteryl sulphate marketed by the company Seporga under the trade name Phytocohesine®; and the extract of maize marketed by the company Solabia under the trade name Phytovityl®.

The myorelaxants which can be used in the composition according to the invention comprise calcium inhibitors such as alverine and its salts, manganese gluconate, chloride channel openers such as diazepam, and inhibitors of catecholamines and of acetylcholine, such as the hexapeptide argireline R marketed by the company Lipotec.

The term “tensioning agent” is intended to mean a compound capable of exerting tension on the skin, the effect of which is to temporarily fade out irregularities on the skin's surface, such as wrinkles and fine lines.

Among the tensioning agents which can be used in the composition according to the invention, mention may in particular be made of:
(1) synthetic polymers, such as polyurethane latices or acrylic-silicone latices, in particular those described in Patent Application EP-1 038 519, such as a propylthio(polymethyl acrylate), propylthio(polymethyl methacrylate) and propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are in particular sold by the compoany 3M under the trade names VS 80, VS 70 or LO21,
(2) polymers of natural origin, in particular (a) polyholosides, for example (i) in the form of starch derived in particular from rice, from maize, from potato, from cassava, from pea, from Triticum aestivum wheat, from oat, etc, or (ii) in the form of carrageenans, alginates, agars, gellans, cellulose-based polymers and pectins, advantageously as an aqueous dispersion of gel microparticles, and (b) latices consisting of shellac resin, sandarac gum, dammar resins, elemi gums, copal resins, cellulose-based derivatives, and mixtures thereof,
(3) plant proteins and protein hydrolysates, in particular from maize, rye, Triticum aestivum wheat, buckwheat, sesame, spelt, pea, bean, lentil, soybean and lupin,
(3) mixed silicates, especially phyllosilicates and in particular laponites,
(4) wax microparticles chosen, for example, from carnauba wax, candelilla wax and esparto wax,
(5) colloidal particles of mineral filler with a number-average diameter of between 0.1 and 100 nm, preferably between 3 and 30 nm, chosen, for example, from: silica, cerium oxide, zirconium oxide, alumina, calcium carbonate, barium sulphate, calcium sulphate, zinc oxide and titanium dioxide.

The term “anti-pollution agent” is intended to mean any compound capable of trapping ozone, monocyclic or polycyclic aromatic compounds such as benzopyrene and/or heavy metals such as cobalt, mercury, cadmium and/or nickel. The term “free-radical scavenger” is intended to mean any compound capable of trapping free radicals.

As ozone-trapping agents which can be used in the composition according to the invention, mention may be made in particular of phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chlorogenic acid; stilbenes, in particular resveratrol; sulphur-containing amino acid derivatives, in particular S-carboxymethylcysteine; ergothioneine; N-acetylcysteine; chelating agents such as N,N′-bis(3,4,5-trimethoxybenzyl) ethylenediamine or one of its salts, metal complexes or esters; carotenoids such as crocetin; and various starting materials, such as the mixture of arginine, histidine ribonucleate, mannitol, adenosine triphosphate, pyridoxine, phenylalanine, tyrosine and hydrolyzed RNA marketed by Laboratoires Sérobiologiques under the trade name CPP LS 2633-12F®, the water-soluble fraction of maize marketed by the company Solabia under the trade name Phytovityl®, the mixture of extract of fumitory and of extract of lemon marketed under the name Unicotrozon C-49® by the company Induchem, and the mixture of extracts of ginseng, of apple, of peach, of wheat and of barley, sold by the company Provital under the trade name Pronalen Bioprotect®.

As agents for trapping monocyclic or polycyclic aromatic compounds, which can be used in the composition according to the invention, mention may in particular be made of tannins such as ellagic acid; indole derivatives, in particular 3-indolecarbinol; extracts of tea, in particular of green tea, extracts of water hyacinth or Eichhornia crassipes; and the water-soluble fraction of maize marketed by the company Solabia under the trade name Phytovityl®.

Finally, as heavy-metal-trapping agents which can be used in the composition according to the invention, mention may in particular be made of chelating agents such as EDTA, the pentasodium salt of ethylenediaminetetramethylenephosphonic acid, and N,N′-bis(3,4,5-trimethoxybenzyl) ethylenediamine or one of its salts, metal complexes or esters; phytic acid; chitosan derivatives, extracts of tea, in particular green tea; tannins such as ellagic acid; sulphur-containing amino acids such as cysteine; extracts of water hyacinth (Eichhornia crassipes); and the water-soluble fraction of maize marketed by the company Solabia under the trade name Phytovityl®.

The free-radical scavengers which can be used in the composition according to the invention comprise, besides certain anti-pollution agents mentioned above, vitamin E and its derivatives such as tocopheryl acetate; bioflavonoids; coenzyme Q10 or ubiquinone; certain enzymes such as catalase, superoxide dismutase, lactoperoxydase, glutathione peroxydase and quinone reductases; glutathione; benzylidenecamphor; benzylcyclanones; substituted naphthalenones; pidolates; phytantriol; gamma-oryzanol; lignans; and melatonin.

The composition according to the invention can be applied to the skin, body hair, the eyelashes, the hair, the nails or the lips, according to the use for which it is intended. It is preferably suitable for topical application to the skin. This composition can thus be used in a cosmetic treatment process for the skin, comprising the application of the composition according to the invention to the skin, for example for the purpose of toning it, of regenerating it, of smoothing out the fine lines of the skin, and/or for combating ageing of the skin and the harmful effects of UV radiation, and/or for strengthening cutaneous tissues against environmental attacks.

In a variant, the composition according to the invention can be used for manufacturing a dermatological preparation, such as a preparation intended to depigment the skin, body hair and/or the hair.

The examples which follow serve to illustrate the invention without, however, being limiting in nature. The compounds are, according to the case, mentioned in terms of chemical names or of CTFA names (International Cosmetic Ingredient Dictionary and Handbook).

### EXAMPLE 1

### Water-in-oil Emulsion

| | | |
|---|---|---|
| Phase A: | Isohexadecane | 8 g |
| | Squalane | 3.7 g |
| | Polydimethylsiloxane | 4.1 g |
| | Apricot kernel oil | 2.3 g |
| | Diethylethanolamine salt of | 1.9 g |
| | polyisobutylene containing an | |
| | esterified succinic end group* | |
| Phase B: | Ascorbic acid | 2 g |
| | 50% potassium hydroxide | 1.2 g |
| | Demineralized water | 67.8 g |
| | Glycerol | 5 g |
| | Preserving agent | 1 g |
| Phase C: | Nylon-12 powder | 3 g |

*marketed under the reference Lubrizol 5603 ® by the company Lubrizol.

Procedure: Phase B is emulsified slowly in phase A at ambient temperature. Phase C is then added.

### COMPARATIVE EXAMPLE 1

### Water-in-oil Emulsion

| | | |
|---|---|---|
| Phase A: | Cyclopentasiloxane and | 20 g |
| | PEG/PPG-18/18 dimethicone* | |
| | Phenyl trimethicone | 7 g |
| | Apricot kernel oil | 5.5 g |
| | Nylon-12 powder | 3 g |
| Phase B: | Ascorbic acid | 2 g |
| | 50% potassium hydroxide | 1.2 g |
| | Demineralized water | 55.3 g |
| | Glycerol | 5 g |
| | Preserving agent | 1 g |

*marketed under the reference DC-2 5225 C by the company Dow Corning.

Procedure: Phase B is emulsified slowly in phase A, at ambient temperature.

### EXAMPLE 2

### Accelerated Storage Test

The aim of this test is to study the decomposition of an oxidation-sensitive hydrophilic active principle in a composition, after storage for two months at 45° C.

The degree of decomposition measured is given by the ratio:
(C₀-C₂ months)/C₀
with C₀ being the concentration of ascorbic acid at t=0 and C₂ months being the concentration of ascorbic acid at t=2 months.

The concentration of ascorbic acid is determined by the HPLC technique (Merck LaChrom system). The analytical conditions are as follows:
Column: Lichrosphere 100 RP18 (250 mm)
Eluant: 0.1M phosphate buffer, pH 2.1
Flow rate: 1 ml/min
Detection at 257 nm.
Dilution of the sample such that the concentration of ascorbic acid is between 0.05 and 1 mg/ml.

The test was carried out with the composition Example 1, and with the composition of Comparative Example 1 which contains, instead of the polymer according to the invention, a conventionally used emulsifier, cyclopentasiloxane and PEG/PPG-18/18 dimethicone.

The results obtained are given in the following table:

Degree of decomposition after 2
months at 45° C. (in %)

| | Under air, amber | Under nitrogen, |
|---|---|---|
| | glass bottle | aluminium flask |
| Example 1 | 7.8 | 3.4 |
| Comparative | 14.4 | 9.8 |
| Example 1 | | |

The results above show that the stability of the ascorbic acid when it is formulated in the presence of diethylethanolamine salt of polyisobutylene containing an esterified succinic end group (Lubrizol 5603) is increased by 50%, even in the presence of oxygen.

### EXAMPLE 3

### Water-in-oil Emulsion

| | | |
|---|---|---|
| Phase A: | Isononyl isononanoate | 4.65 g |
| | Plant perhydrosqualene | 3.48 g |
| | Polydimethylsiloxane (viscosity 10 cst) | 2.33 g |
| | Diethylethanolamine salt of | 1.92 g |
| | polyisobutylene containing an | |
| | esterified succinic end group* | |
| Phase B: | Ascorbic acid | 5 g |
| | 50% potassium hydroxide | 3.17 g |
| | Demineralized water | 67.95 g |
| | Glycerol | 5 g |
| | Preserving agents | 1 g |
| | Ammonium polyacryloyldimethyl | 0.2 g |
| | taurate | |
| | Magnesium sulphate.7H₂O | 2 g |
| Phase C: | Silica microspheres | 3 g |
| Phase D: | Fragrance | 0.3 g |

*marketed under the reference Lubrizol 5603 ® by the company Lubrizol.

Procedure: Phase B is emulsified in phase A, at ambient temperature, and then phases C and D are successively introduced. A soothing white cream which is fresh on application, which tones the skin, and in which the ascorbic acid exhibits good stability, is obtained.

The above written description of the invention provides a manner and process of making and using it such that any person skilled in this art is enabled to make and use the same, this enablement being provided in particular for the subject matter of the appended claims, which make up a part of the original description and including a composition for topical use, comprising, in a physiologically acceptable medium, at least one hydrophilic active principle selected from the group consisting of ascorbic acid and its derivatives, and at least one amphiphilic polymer selected from the group consisting of the oligomers or polymers derived from polyisobutylene comprising an apolar polyisobutylene component comprising at least 40 carbon atoms, and at least one terminal polar component consisting of carboxylic acids or diacids, of anhydrides thereof or of modified forms thereof in the form of esters, of amides or of salts, and mixtures thereof, said composition being devoid of N-vinylimidazole polymer or copolymer. Preferred embodiments of the invention similarly fully described and enabled include the use of an amphiphilic polymer selected from the group consisting of the oligomers or polymers derived from polyisobutylene comprising an apolar polyisobutylene component comprising at least 40 carbon atoms, and at least one terminal polar component consisting of carboxylic acids or diacids, of anhydrides thereof or of modified forms thereof in the form of esters, of amides or of salts, and mixtures thereof, for stabilizing an oxidation-sensitive hydrophilic active principle. Similarly enabled is use of the invention composition to tone the skin, to regenerate the skin and/or to smooth out the fine lines of the skin, and/or for combating ageing of the skin and/or combating the harmful effects of UV radiation, and/or for strengthening cutaneous tissues against environmental attacks, for manufacturing a preparation intended to depigment the skin, body hair and/or the hair.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

As used herein, where a certain polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted and reactive “monomers,” but instead is made up of repeating units derived from reacted monomers.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising:
a physiologically acceptable medium,
at least one ascorbic acid compound, and
at least one amphiphilic polyisobutylene polymer, said polymer comprising an apolar polyisobutylene component comprising at least 40 carbon atoms and at least one terminal polar component selected from the group consisting of carboxylic acids, carboxylic diacids, anhydrides of carboxylic acids, and anhydrides of carboxylic diacids, esters thereof, amides thereof, salts thereof, and mixtures thereof,
wherein said composition is devoid of N-vinylimidazole polymer or copolymer.

2. The composition according to claim 1, comprising at least one of 5,6-di-O-dimethylsilylascorbate, the potassium salt of dl-alpha-tocopheryl-dl-ascorbyl phosphate, magnesium ascorbyl phosphate, sodium ascorbyl phosphate, and ascorbyl glucoside.

3. The composition according to claim 1, comprising ascorbic acid.

4. The composition according to claim 1, wherein the at least one ascorbic acid compound is present in an amount of 0.5-20% by weight relative to the total weight of the composition.

5. The composition according to claim 1, wherein the apolar polyisobutylene component of the amphiphilic polyisobutylene polymer comprises from 60 to 700 carbon atoms.

6. The composition according to claim 1, wherein the terminal polar component is selected from the group consisting of carboxylic diacids, anhydrides of carboxylic diacids, esters of diacids, amides of diacids, salts thereof, and mixtures thereof.

7. The composition according to claim 1, wherein the amphiphilic polyisobutylene polymer is derived from the reaction between a derivative of polyisobutylene and at least one acid or anhydride selected from the group consisting of succinic acid, succinic anhydride, maleic acid, maleic anhydride, fumaric acid, itaconic acid, citraconic acid, mesaconic acid, aconitic acid, modified forms thereof in the form of esters, of amides or of salts, and mixtures thereof.

8. The composition according to claim 1, wherein the amphiphilic polyisobutylene polymer is a polyisobutylene comprising a modified succinic end group.

9. The composition according to claim 1, wherein the amphiphilic polyisobutylene polymer is the product of the reaction of maleic anhydride with polyisobutylene.

10. The composition according to claim 1, wherein the amphiphilic polyisobutylene polymer is the diethylethanolamine salt of polyisobutylene containing an esterified succinic end group.

11. The composition according to claim 1, wherein the amount of amphiphilic polyisobutylene polymer is 0.1-10% by weight relative to the total weight of the composition.

12. The composition according to claim 1, wherein said composition is in the form of a water-in-oil emulsion.

13. The composition according to claim 1, wherein the molar ratio of the amphiphilic polyisobutylene polymer to the at least one ascorbic acid compound is 0.0006-3.

14. The composition according to claim 1, wherein the at least one amphiphilic polyisobutylene polymer is the only emulsifier present in the. composition.

15. A method of stabilizing an oxidation-sensitive hydrophilic active principle, comprising mixing said oxidation-sensitive hydrophilic active principle with at least one amphiphilic polyisobutylene polymer, said polymer comprising an apolar polyisobutylene component comprising at least 40 carbon atoms and at least one terminal polar component selected from the group consisting of carboxylic acids, carboxylic diacids, anhydrides of carboxylic acids, and anhydrides of carboxylic diacids, esters thereof, amides thereof, salts thereof, and mixtures thereof.

16. The method according to claim 15, wherein the oxidation-sensitive hydrophilic active principle is ascorbic acid.

17. A process, comprising applying to the skin the composition according to claim 1.

18. A process to tone the skin, and/or to regenerate the skin and/or to smooth out the fine lines of the skin, and/or for combating the appearance of ageing of the skin and/or combating the harmful effects of UV radiation, and/or for strengthening cutaneous tissues against environmental attacks, comprising applying to the skin an effective amount of the composition according to claim 1 to tone the skin, and/or to regenerate the skin, and/or to smooth out the fine lines of the skin, and/or to combat the appearance of ageing of the skin, and/or to combat the harmful effects of UV radiation, and/or to strengthen cutaneous tissues against environmental attacks.

19. A method as claimed in claim 18, wherein the composition comprises at least one ascorbic acid compound in an amount of 0.5-20% by weight relative to the total weight of the composition, the amount of amphiphilic polyisobutylene polymer is 0.1-10% by weight relative to the total weight of the composition, and wherein the amphiphilic polyisobutylene polymer is selected from the group consisting of a polyisobutylene comprising a modified succinic end group, the product of the reaction of maleic anhydride with polyisobutylene, the diethylethanolamine salt of polyisobutylene containing an esterified succinic end group, and mixtures thereof.

20. A method to depigment the skin, body hair and/or the hair, comprising applying thereto the composition of claim 1.
